(19) United States
(12) Patent Application Publication
(10) Pub. No.: US 2024/0399101 A1
(43) Pub. Date: Dec. 5, 2024

(54) DEVICE AND METHOD FOR MODIFYING AN EMOTIONAL STATE OF A USER

(71) Applicant: APHELIOR, LILLE (FR)

(72) Inventor: Athenais OSLATI, LILLE (FR)

(21) Appl. No.: 18/262,746

(22) PCT Filed: Feb. 23, 2022
(86) PCT No.: PCT/EP2022/054518

§ 371 Date: Jul. 25, 2023

(30) Foreign Application Priority Data
Feb. 23, 2021 (FR) FR2101749

(51) Int. Cl. A61M 21/00; G06F 3/16; G10H 1/00; H04R 1/10

(52) U.S. Cl. CPC A61M 21/00; G06F 3/165; G10H 1/0008; A61M 2021/0027; A61M 2209/088; A61M 2230/10; G10H 2210/031; G10H 2250/311; H04R 1/1041

(57) ABSTRACT

This device (100) for modifying an emotional state of a user comprises:
a real-time reader (105) of electroencephalographic signals;
   a module (110) for determining an emotional state based on an electroencephalographic signal read;
a means (115) for determining a target emotional state;
an automatic selector (120) of a sequence of at least one sound file, from a previously assembled list of sound files, based on the target emotional state determined, the electroencephalographic signal read, and at least one parameter associated to each said sound file;
an electroacoustic transducer (125) configured to play the selected sequence of sound files;
a secondary selector (130) of a sound file; and
a means (135) for updating the sequence based on the sound file selected manually by the secondary selector.

### TECHNICAL FIELD OF THE INVENTION

The present invention relates to a device and method for modifying an emotional state of a user. It applies, in particular, to the field of improving the well-being and controlling the emotional state of an individual.

### STATE OF THE ART

The development of cognitive, motor and sensory abilities is part of a wish to increase the human life expectancy. In this search for performance and well-being, neuro-technology is a key solution. This research field is the result of the convergence of neurosciences and computing that is at the root of changes to mental models and maps.

One of the goals of neuro-technologies is to increase human performance and improve their well-being. Such increases and improvements are possibly achieved by modifying, for example, the emotional stress of the human being.

Emotional stress is a normal, non-pathological, reaction of the human body faced with environmental stimuli (stressors), for example. Such a stress is therefore an archaic natural defence mechanism that can apply to all human beings, without being associated with an illness.

Currently, the following solutions are known:
Melomind (registered trademark): a system comprising an audio headset delivering sounds in order to directly monitor an individual's level of relaxation;
   Muse (registered trademark): a system comprising EEG (for “electroencephalogram”) sensors measuring tiny electrical fields in the brain, and heart rate, respiration and oximetry sensors;
Dreem-Urgo (registered trademark): a system comprising a physiological data sensor and EEG sensors; and
Emotiv (registered trademark): a system comprising an EEG sensor and earphones enabling an indication of the user's level of stress and distraction.

However, all these solutions have the following drawbacks:
The user's preferences regarding sound stimuli are not taken into account: The algorithms for the provision of sound stimuli only take the individual's brain activity into consideration, imposing noises that the individual is not accustomed to, resulting in the individual's disinterest in, even rejection of, the solutions;
No flexibility in real time: although these technologies collect the signals of brain activity in real time, the algorithms used cannot respond to unanticipated variations in the user's wishes during his uses. They are designed so that a single route, very often based on the user's brain activity and/or physiology (heart rate), takes the user from point A to point B;
Extremely limited emotional trajectories: Currently, the user cannot target the emotional state he wants to reach. Indeed, if he purchases a Melomind or Muse headset, it is only to relax. While some systems, such as Emotiv, enable different emotions to be read, they do not make it possible to take the user from one emotion to another. These technologies are often one-way. They start from stress to approach a state of relaxation or concentration. The inverse is therefore not possible.

Document CN 110 947 076 is known, which discloses a smart portable device of brain-wave music for regulating the mental state. Document US 2019/060 605 is also known, which discloses a device for modifying the user's cognitive state. However, neither of these devices enables the sound files played to be adjusted in real time according to a user's choices.

In addition, document US 2018/027 347 is known, which discloses a sound analysis system for automatically predicting the effect these sounds will have on the user. However, this device does not enable the combination of a real-time detection of the user's emotional state and a real-time adjustment, based on a user's choices, of the sound files played.

### Subject of the Invention

The present invention aims to remedy all or part of these drawbacks.

To this end, according to a first aspect, the present invention envisions a device for modifying an emotional state of a user, which device comprises:
a real-time reader of electroencephalographic signals;
   a module for determining an emotional state based on an electroencephalographic signal read;
a means for determining a target emotional state;
   an automatic selector of a sequence of at least one sound file, from a previously assembled list of sound files, as a function of the target emotional state determined, the electroencephalographic signal read, and at least one parameter associated to each said sound file;
an electroacoustic transducer configured to play the selected sequence of sound files;
a secondary selector of a sound file; and
a means for updating the sequence based on the sound file selected manually by the secondary selector.

Thanks to these provisions, a user can voluntarily identify an emotional state to be reached, the device determining an optimum sound files vector and playing each sound file in sequence to gradually adjust the user's emotional state.

The use of a real-time reader of electroencephalographic signals enables the sequence to be adjusted dynamically based on its success in modifying the user's emotional state or his actions, such as, for example, the unanticipated playing of a sound file requiring an update to the sound files vector.

The use of a previously assembled list of sound files, in particular produced by artists favoured by the user, makes it possible to minimise the risk of the user rejecting the device.

In addition, these embodiments allow the user to voluntarily select a sound file to be played, interrupting the sequence selected, while enabling the device to adjust the sound files sequence to this interruption.

In some embodiments, the device that is the subject of the present invention comprises:
a collector of sound file identifiers; and
a sound file classifier configured to associate, to at least one sound file identifier, a parameter representative of an emotional state fostered by that sound file.

These embodiments make it possible to associate, to sound files in a previously assembled list, indicators used in the selection of sound files to be played.

In some embodiments, the classifier is a trained machine learning system.

These embodiments enable new sound files to be classified automatically, ensuring efficient updating of the previously assembled list of sound files.

In some embodiments, the trained machine learning system is a supervised neural network configured to receive, as an input layer, parameter values and, as an output layer, emotional state indicators corresponding to the input layer.

In some embodiments, the classifier is configured to classify a sound file by assigning a value to at least one of three characteristics:
valence;
arousal; and
dominance;
   at least one characteristic being utilised to determine an emotional state fostered by a sound track.

In some embodiments, the machine learning system is also pre-trained by using a set of data not specific to the user.

Thanks to these provisions, the machine learning system is pre-trained, for example, prior to the use of the device by external data. The system therefore undergoes additional training. As a result, the pre-training of the learning system is strengthened.

In some embodiments, at least one sound file is associated to an indicator of a behaviour of the user regarding each said sound file, the automatic selector being configured to select a sequence of at least one sound file based on a value of this indicator for at least one sound file.

These embodiments make it possible to quantify the user's preference regarding a sound file so as to minimise the risk of the selected sequence being rejected.

In some embodiments, the indicator of the user's behaviour is a parameter representative of a number of plays and/or of a number of playback interruptions in favour of another sound track.

Thanks to these provisions, the behaviour indicator is easily determined.

In some embodiments, the automatic selector comprises a sound file filter based on at least one indicator of a behaviour of the user regarding at least one sound file, the selector being configured to select a sound files sequence from a list of sound files filtered by the filter.

These embodiments make it possible to quantify the user's preference regarding a sound file so as to minimise the risk of the selected sequence being rejected.

In some embodiments, a parameter used by the automatic selector to select a sound files sequence is an indicator representative of an emotional state value associated to at least one sound file.

In some embodiments, a parameter used by the automatic selector to select a sound files sequence is, in addition, a technical parameter chosen from the duration, mode, tonality, quantification of the beat and the tempo of the sound file.

Thanks to these provisions, a selection made by the automatic selector is based on technical parameters inherent in the sound file. As a result, the automation of the device is increased.

In some embodiments, the sound files sequence is configured to have a gradient of increasing emotional state value corresponding to the target emotional state determined.

These embodiments make it possible to determine a sequence of increasing intensity, associated to a target emotional state.

In some embodiments, the real-time reader of electroencephalographic signals is non-invasive.

In some embodiments, the reader is an electroencephalogram type of headset.

Thanks to these provisions, use of the device is made easier. In addition, the user's bodily integrity is maintained during use of the device. In other words, there is no physical discomfort when the device is used. Additionally, when the reader is a headset, the device can move in line with the user's movements.

According to a second aspect, the present invention envisions a method for modifying an emotional state of a user, which method comprises:
a step of determining a target emotional state;
and then, iteratively, at least one portion of the following steps:
a step of the real-time reading of electroencephalographic signals;
a step of determining an emotional state based on an electroencephalographic signal read;
a step of automatically selecting a sequence of at least one sound file, from a previously assembled list of sound files, based on the target emotional state determined, the electroencephalographic signal read, and at least one parameter associated to each said sound file;
a step of an electroacoustic transducer playing the selected sequence of sound files;
a step of a secondary selection of a sound file; and
a step of updating the sequence based on the sound file selected manually by the secondary selector.

As the particular features, advantages and aims of the method that is the subject of the present invention are identical to those of the device that is the subject of the present invention, they are not repeated here.

### BRIEF DESCRIPTION OF THE FIGURES

Other advantages, aims and particular features of the invention will become apparent from the non-limiting description that follows of at least one particular embodiment of the device and method that are the subjects of the present invention, with reference to drawings included in an appendix, wherein:

FIG. 1 represents, schematically, a first particular embodiment of the device that is the subject of the present invention;

FIG. 2 represents, schematically, a second particular embodiment of the device that is the subject of the present invention;

FIG. 3 represents, schematically and in the form of a logic diagram, a particular series of steps of the method that is the subject of the present invention;

FIG. 4 represents, schematically, a distribution of a sample of music files, in terms of their energising nature, with values between 0.1 and 1;

FIG. 5 represents, schematically, a distribution of a sample of music files, in terms of their dance nature, with values between 0.1 and 1;

FIG. 6 represents, schematically, a distribution of sound files to be played corresponding to a logarithmic vector determined for modifying the emotional state;

FIG. 7 represents, schematically, a distribution of sound files to be played corresponding to a linear vector determined for modifying the emotional state;

FIG. 8 represents, schematically, a distribution of acoustical values of sound files corresponding to different emotional states; and

FIG. 9 represents, schematically, a distribution of sound volume values of sound files corresponding to different emotional states.

### DESCRIPTION OF THE EMBODIMENTS

The present description is given in a non-limiting way, in which each characteristic of an embodiment can be combined with any other characteristic of any other embodiment in an advantageous way.

Note that the figures are not to scale.

The term “emotional state” refers to the results of the interaction of subjective and objective factors, by neural or endocrine systems, which can:
induce experiences such as feelings of alertness, pleasure or displeasure;
   generate cognitive processes such as appropriate reorientations on the perceptual level, evaluations, labelling;
activate overall physiological adjustments;
induce behaviours that are, most frequently, expressive, goal-directed, and adaptive.

For a human being, such an emotional state is, for example:
Stress (difficulty concentrating, thought process chaotic and disturbed by the source of the stress, arousal, fear);
Relaxation (state of calm, lack of or low reflective activity, ideas flow slowly);
Concentration (intense brain activity, unreceptivity to outside signals); and
Interest or engagement (measure of the level of immersion in any stimulus.

Engagement can be increased during immersion in stimuli which may be positive or negative. Experimental brain research has found that engagement declines in cognitive processes that are boring, mundane and automatic).

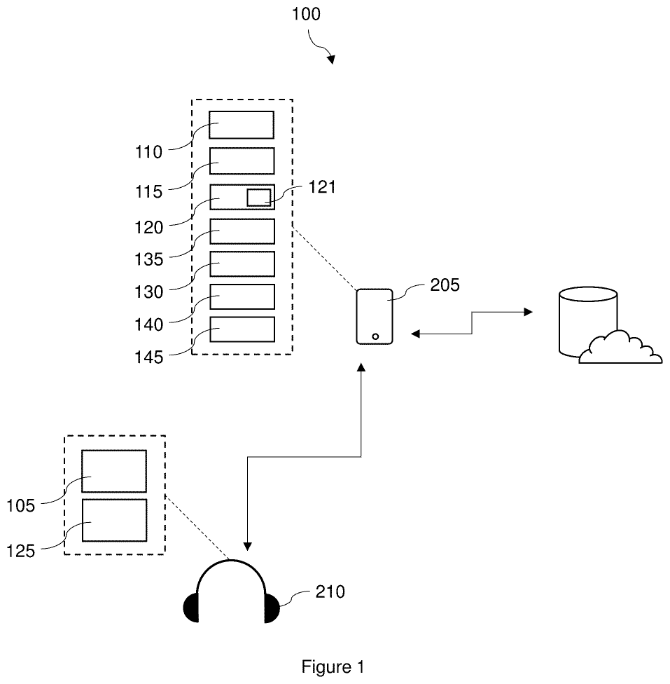

FIG. 1, which is not to scale, shows a schematic view of an embodiment of the device 100 that is the subject of the present invention. This device 100 for modifying an emotional state of a user comprises:
a real-time reader 105 of electroencephalographic signals;
   a module 110 for determining an emotional state based on an electroencephalographic signal read;
a means 115 for determining a target emotional state;
an automatic selector 120 of a sequence of at least one sound file, from a previously assembled list of sound files, based on the target emotional state determined, the electroencephalographic signal read, and at least one parameter associated to each said sound file; and
an electroacoustic transducer 125 configured to play the selected sequence of sound files.

The reader 105 is, for example, an electroencephalogram type of headset equipped with earphones acting as an electroacoustic transducer 125. The type of electroencephalogram considered can be any type known to a competent person in the field of neuro-technologies. Preferably, a non-invasive electroencephalogram is utilised.

The function of an electroencephalogram is to capture electric signals resulting from the summation of synchronous post-synaptic potentials from a large number of neurons. Such a signal can be representative of a neurophysiological activity of the individual wearing the reader 105 and, therefore, of an emotional state of that individual.

Acquisition is made, for example, by means of seven dry electrodes placed on a user's scalp, preferably in positions A1, T3, C3, CZ, C4, T4, A2, T5 and T6 according to the 10-20 system. These electrodes measure the potential difference (in Volts) between the various positions and “the earth”, placed on the closest ear.

The choice of the position of these electrodes is mainly linked to the geometry of the headset and to comfort of use, but also to the selection of certain points less subject to motor artefacts (blinking of the eyelids, sniffing, etc.). The electrodes are connected to a digital card. This card is configured to transmit the signal to the determination module 110 or to a computer, for example via Bluetooth to a USB receiver plugged into the computer.

The sampling interval chosen is, for example, 5 seconds (every 5 seconds, the signal for the last 5 seconds is received). This value can be increased to 8 seconds, even 10 seconds, 5, 8 and 10 being the sampling intervals allowing the best inference of emotions.

From the raw signal, the intensity of the different frequencies can be calculated by Fourier transformation. A first signal pre-processing can be applied to remove the frequencies close to 50 Hz (49-51 Hz) or 60 Hz (59-61 Hz), which are intensely parasitised in the presence of electric appliances plugged into the electric grid near the recording appliance (the headset).

A second bandpass filter can be applied to retain only the frequencies in the range 2-58 Hz, in order to eliminate the low-frequency parasitic noise, and the high-frequency gamma bands (60 Hz and higher) that Bluetooth does not enable us to describe properly.

The two filters used are, for example, 5th-order Butterworth type.

The determination module 110 is, for example, a computer system running on an electronic calculation circuit. This determination module 110 is configured, for example, to determine an emotional state of the individual as follows:

The model chosen to describe the emotions is the commonly used three-variable system called the “VAD” (for the Valence, Arousal and Dominance axes) model.

Valence describes the negative, neutral or positive character associated with an emotion.

Arousal measures the passive, neutral or active character of the emotional state described.

Dominance describes the dominant or submissive character of the emotional state described. This axis makes it possible, for example, to distinguish rage from fear (both of which are characterised by low valence and high arousal), or relaxation from joy.

For each axis, 3 possible discrete values are defined:
Valence: −1 for negative, 0 for neutral, 1 for positive;
Arousal: −1 for passive, 0 for neutral, 1 for active; and
Dominance: −1 for low, 0 for medium, 1 for high.

The following labels are assigned to the V-A-D value triplets:
Excited: (1,1,1)
Happy: (1,0,1)
Content: (1,0,0)
Relaxed, Mellow: (1,−1,0)
Calm: (0,−1,0)
Sad, Depressed: (−1,−1,−1)
De-stressed: (0,1,0)
Neutral: (0,0,0)
Deep sadness: (−1,0,−1)

Other emotional descriptors can be used in addition to the VAD values, because easy to detect and recognise from brain recordings, such as relaxation and concentration.

Each sound track can be classed according to these coordinates for a step-by-step vector calculation making it possible, based on coordinates determined, to reach other coordinates corresponding to a target emotional state.

The determination module 110 can be implemented locally or remotely and accessible via a data network. For example, the determination module 110 can be implemented on a smartphone having a wired or, preferably, wireless connection to the reader 105.

The means 115 for determining a target emotional state is, for example, a human-machine interface (for example, a graphic interface associated to an input device) or a software interface (for example, an API, for Application Programming Interface). This determination means 115 is configured to receive, on input, a signal variable between several possible emotional states.

These emotional states can be pre-determined, i.e. forming a finite list of possibilities from which an implemented interface makes a selection.

These emotional states can be determined based on the content of an entry made via an interface. For example, a human-machine interface allows the free entry of alphanumeric characters representative of a human language, a user of the device 100 entering descriptor keywords for an emotional state to be reached, the determination means 115 being configured to associate defined emotional states to these keywords.

The determination means 115 can be implemented locally or remotely and accessible via a data network. For example, the determination means 115 can be implemented on a smartphone associated to the determination module 110.

The automatic selector 120 is, for example, a computer system running on an electronic calculation circuit. The automatic selector 120 is configured, for example, to execute an algorithm measuring a distance between the emotional state read of a user of the device 100 and the target state determined by the determination means 115.

According to the distance measured in this way, a sequence of at least one sound file is selected based on at least one parameter associated to each said sound file.

Such a parameter can, for example, be:
a technical parameter, such as the duration, mode, tonality, quantification of the beat or tempo of the sound file; or
an acoustic or psychoacoustic parameter representative of:
the acoustic nature of the sound file i.e. whether or not electronic instruments are used and/or the proportion of their use;
the energising, or invigorating, nature of the sound file, i.e. a perceptive measurement of the intensity and activity—the perceptual characteristics contributing to this attribute comprise the dynamic range, the perceived sound intensity, the timbre, the rate of occurrence and the general entropy;
the instrumental nature of the sound file, i.e. whether or not the voice is used in this sound file;
the dance nature of the sound file, measured, for example, based on the tempo, stability of the rhythm, strength of the beat and the general regularity of the audio file;
the valence of the sound file, i.e. the positivity of the sound file;
the nature of the sound file recording, i.e. sound file recorded in a studio or during a direct performance;
the nature of the density of words, i.e. the proportion of words and music in a sound file; and/or
the intensity of the sound file, i.e. the average intensity, measured in decibels, of the sound file.

Each of these parameters can be directly associated to an emotional state and therefore, depending on the target emotional state, be a candidate for inclusion in the sound files sequence.

The association between values for these parameters and emotional states (via, for example, a V-A-D profile) can be carried out, for example, by utilising a learning algorithm, obtained in a similar way to the IADS-E dataset of the Center for the Study of Emotion and Attention at the University of Florida.

Alternatively, an expert system can be utilised, associating specific values of the V-A-D profile to an emotional state. An example of such a utilisation is provided above.

In a simplified mode, the energising nature and the dance nature are associated to the arousal, the mode and valence with the valence, and the intensity with the dominance.

For each sample, one then constructs, for example, a statistical model of each acoustic descriptor. The model chosen is, for example, a Gaussian mixture, i.e. a weighted set of one to five Gauss curves (“bell curves”) whose means and standard deviations are recorded, and also the weights associated to each Gaussian. The mixed Gaussian model obtained describes a probability density curve, which associates to each value of the acoustic parameter considered the probability of being observed for an audio track of the given group (high or low valence, high or low arousal, high or low domination).

This gives an approximation of the probability that an audio track with given acoustic characteristics is in each quadrant of the VAD space.

One calculates the mean, on each axis and for each audio track, of the probability of belonging to the positive and negative quadrant. In this way one obtains the coordinates of a sound file in question in the VAD space. This position in the VAD space will be read when the audio tracks to be added to a playlist is determined.

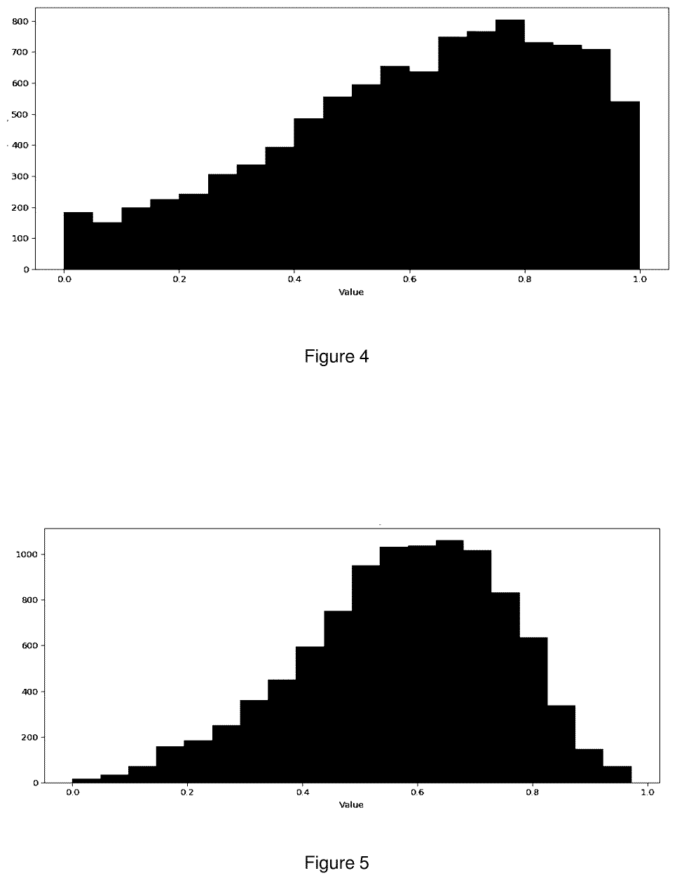

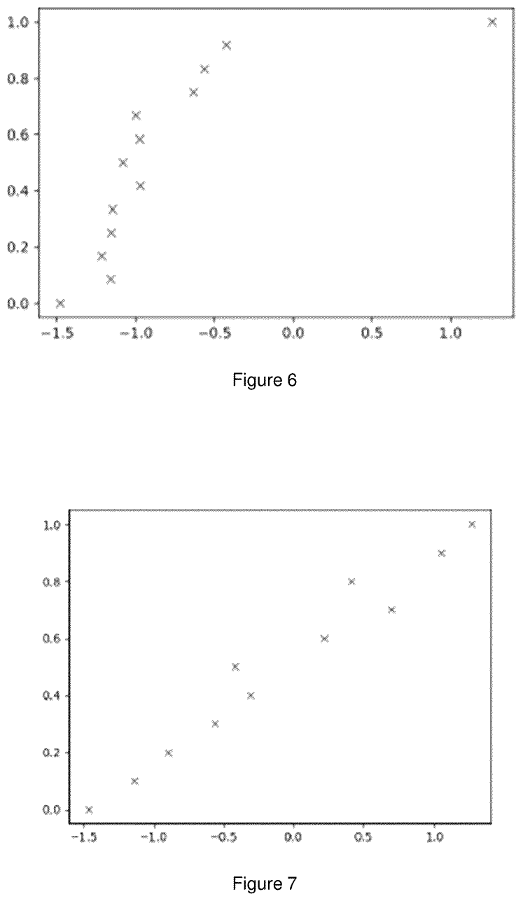

FIG. 4 shows, schematically, a distribution of a sample of music files, in terms of their energising nature, with values between 0.1 and 1.

FIG. 5 shows, schematically, a distribution of a sample of music files, in terms of their dance nature, with values between 0.1 and 1.

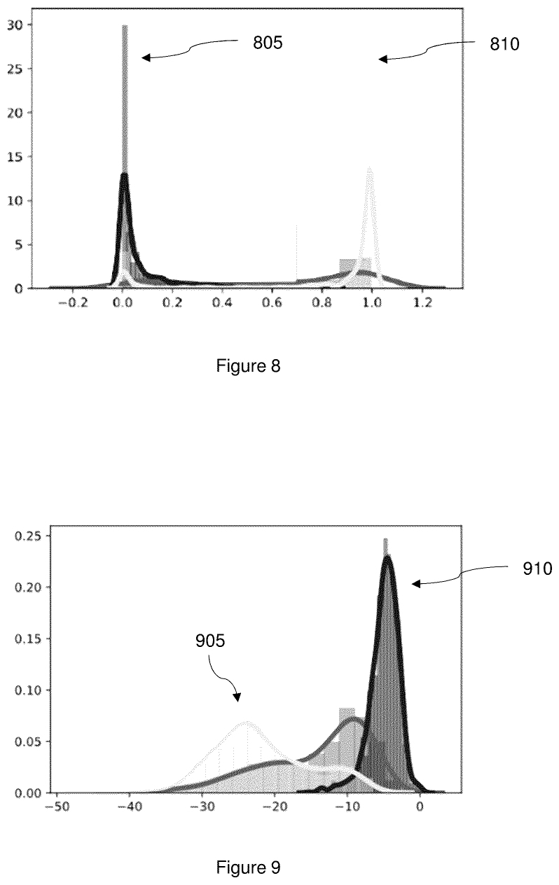

FIG. 8 shows, schematically, a distribution of the quantification of the acoustic nature of a sample of sound files corresponding, in particular, to two different states:
an emotional state of calm 805 and
an emotional state of concentration 810.

FIG. 9 shows, schematically, a distribution of the quantification of the sound volume of a sample of sound files corresponding, in particular, to two different states:
an emotional state of concentration 905 and
an emotional state of intense physical activity 910.

Preferably, a parameter used by the automatic selector 120 to select a sound files sequence is an indicator representative of an emotional state value associated to at least one sound file. Each sound file is then associated to a vector quantifying the impact for at least one emotional state. For example, a sound file can have a first value corresponding to the impact of this sound file on a listener's level of stress, and a second value corresponding to the impact of this sound file on a listener's level of relaxation.

Preferably, the sound files sequence is configured to have a gradient of increasing emotional state value corresponding to the target emotional state determined.

In other words, the actual and target emotional states determined are described by the coordinates on axes that all or part of the parameters listed above constitute, in a multi-dimensional space.

Such a vector can be constituted from at least one of the parameters described above.

Such a vector, in a defined dimensional space, can correspond to a linear or logarithmic function.

According to a first algorithm, a theoretical straight-line path between the two points in the VAD space is calculated first. It does not yet correspond physically to a list of sound files. Next, the algorithm samples points spaced regularly along this theoretical line (based on the number of sound files desired, itself a function of the desired duration of the playlist). Lastly, the sound files in the database whose coordinates in this space are the closest to each of the theoretical points are selected, which results in an ordered list of sound files.

According to a second algorithm, the selection of audio tracks is performed iteratively, by systematically searching for the file closest to the median point between the bounds of the search interval. First of all, the theoretical median point between the two points (actual and target states) is calculated. Next, the sound file in the database that is closest to this point in the VAD space is determined, using its coordinates. This file makes it possible to cut the interval in two and produce two new intervals, on which the procedure is repeated until, optionally, a maximum number of audio tracks is obtained.

The final path obtained is less linear than with algorithm 1, but allows smoother transitions between the tracks.

The automatic selector 120 can be implemented locally or remotely and accessible via a data network. For example, the automatic selector 120 can be implemented on a smartphone associated to the determination means 115 and the determination module 110.

The sound files sequence is sent to an electroacoustic transducer 125. This transmission can be performed through the motherboard or a sound card of a smartphone interfaced with the automatic selector 120.

In some particular embodiments, such as that shown in FIG. 1, the device 100 that is the subject of the present invention comprises:
a collector 140 of sound file identifiers; and
a sound file classifier 145 configured to associate, to at least one sound file identifier, a parameter representative of an emotional state fostered by that sound file.

The collector 140 of identifiers is, for example, a computer system running on an electronic calculation circuit. This collector 140 is, for example, configured to collect the identifiers of sound files whose playing is controlled by at least one user associated to the device 100 through a third-party application for playing sound files. In some variants, the collector 140 of identifiers is a software system for reading metadata of sound file identifiers stored in a local or remote computer storage.

The collector 140 of identifiers can be implemented locally or remotely and accessible via a data network.

The classifier 145 is, for example, a computer system running on an electronic calculation circuit. This classifier 145 is configured to assign, based on parameters of the sound files, as described above, a quantitative value of the impact of the sound file on the emotional state of a listener to the sound file.

In some particular embodiments, the classifier 145 is a trained machine learning system. Such a classifier 145 can be, for example, a machine learning algorithm, supervised or not, of type deep learning or not.

For example, such a machine learning system is a supervised neural network device configured to receive, as an input layer, parameter values as mentioned above and, as an output layer, emotional state indicators corresponding to the input layer.

In some embodiments, the classifier 145 is configured to classify a sound file by assigning a value to at least one among three characteristics:
valence;
arousal; and
dominance;
at least one characteristic being utilised to determine an emotional state fostered by a sound track. Optionally, concentration and/or relaxation can receive a value, via the classifier 145 or another assignment mechanism.

In an example of implementation consisting of producing such a classifier 145, a computer program allows the user to report when he deeply feels, in his body, that he is in one of the emotional states listed above. If he confirms his state, the sample recorded is sent to the classification model, which reinforces its learning.

The frequent use of this reinforcement tool is necessary for the model to learn properly. Until this tool has been used sufficiently by the user (several tens of times with representation of all the emotions), the performance of the model goes from random, to poor, then mediocre, and finally acceptable. A pre-trained classifier 145 can be implemented by using a set of data not specific to the user, to have correct performance from the start.

The model is trained to recognise an emotion, not from the raw signal but from transformations of it, called characteristics that are calculated from a sample recorded during a time t, on the 7 channels listed above:

For each channel:
the spatial characteristics: the intensity of the alpha [7-13 Hz], beta [14-30 Hz] and gamma [31-90 Hz] frequency bands obtained by Fourier transformation, and the differential entropy; and
the temporal characteristics: the approximate entropy, the sample entropy, and the fractal dimension.

By taking all the channels into account:
the multi-scale characteristics (Multi-Scale-Entropy features); and
the Renyi entropy (or, more specifically, a non-parametric estimation of the Renyi entropy).

A classification algorithm can be an ensemble method (a method that averages the prediction of several classifiers) called “Random Forest”, the classifiers used being decision trees. A population of one hundred decision trees is used, for example.

A decision tree is a series of rules that use thresholds for the values of the characteristics.

The algorithm's training phase consists of varying these thresholds until an acceptable prediction quality is obtained. Each new sample obtained for which the associated emotional state is known makes it possible to refine the thresholds a bit more.

The performance of the models varies from one individual to the next, and cannot be estimated in a general way. The scientific literature declares averages fluctuating between 60% and 85% for correct predictions, depending on individuals.

Therefore, the computer program that forms the training means carries out the following steps:
receive the raw signal from the EEG headset (5-second sample);
calculate the characteristics of the signal;
estimate the valence, arousal, and domination values of the user's actual emotional state, by using a means for predictions via decision trees.
transform the coordinates (V, A, D) obtained into a percentage of each emotion whose label is known, by calculating the Euclidean distance to the position of these known emotions in the VAD space and to a normalised inverse of these distances,
if the user reports, via an input device (keyboard, touch screen), that he is in a known emotional state, propose using the most recent sample to take part in training the model and, if confirmed, adjusts the thresholds of the decision trees using this new sample; and
wait until a new sample is received.

When the training program is halted, the population of decision trees is backed up, to be loaded at the next startup.

In some particular embodiments, at least one sound file is associated to an indicator of a behaviour of the user regarding each said sound file, the automatic selector 120 being configured to select a sequence of at least one sound file based on a value of this indicator for at least one sound file.

Such an indicator of behaviour is, for example, a parameter representative of a number of plays, a number of playback interruptions in favour of another sound track or any other parameter representative of a wish of the user. This indicator of behaviour can be utilised in the selection of the sound files sequence, for example by assigning a lower weight to the candidate sound files having a higher-than-average number of playback interruptions, reflecting the user's dissatisfaction when these files are played.

In some embodiments, at least one parameter used by the automatic selector 120 is a parameter representative of a musical similarity between sound files. Such a musical similarity can be established, for example, based on a metadata representative of a musical genre or based on the parameters described above, with regard to the automatic selector 120.

A musical similarity is determined based on Euclidean distances in the space of the parameters (normalised by their units) exemplified above.

In some particular embodiments, such as that shown in FIG. 1, the automatic selector 120 comprises a sound file filter 121 based on at least one indicator of a behaviour of the user regarding at least one sound file, the selector being configured to select a sound files sequence from a list of sound files filtered by the filter.

Such a filter 121 is, for example, a software filter making it possible to establish a sample of candidate sound files to be selected in the sound files sequence prior to the actual selection.

In some particular embodiments, such as that shown in FIG. 1, the device 100 that is the subject of the present invention also comprises a secondary selector 130 of a sound file and a means 135 for updating the sequence based on the sound file selected manually by the secondary selector.

The secondary selector 130 is, for example, a human-machine interface (for example, a graphic interface associated to an input device) or a software interface (for example, an API). This secondary selector 130 is configured to receive, on input, a sound file identifier for playing. One variant of such a secondary selector 130 is, for example, a touch screen associated to a Graphic User Interface (“GUI”) enabling a sound file identifier to be entered.

This selector 130 allows a user to force the playing of a sound file, irrespective of this sound file's beneficial or negative effect on the target emotional state determined.

However, the quantification of this beneficial or negative effect enables the determination means 115 to determine a new sequence of sound files making it possible to reach the subsequent target emotional state based on the deviation caused by playing the sound file selected by the secondary selector 130.

The secondary selector 130 can be implemented locally or remotely and accessible via a data network.

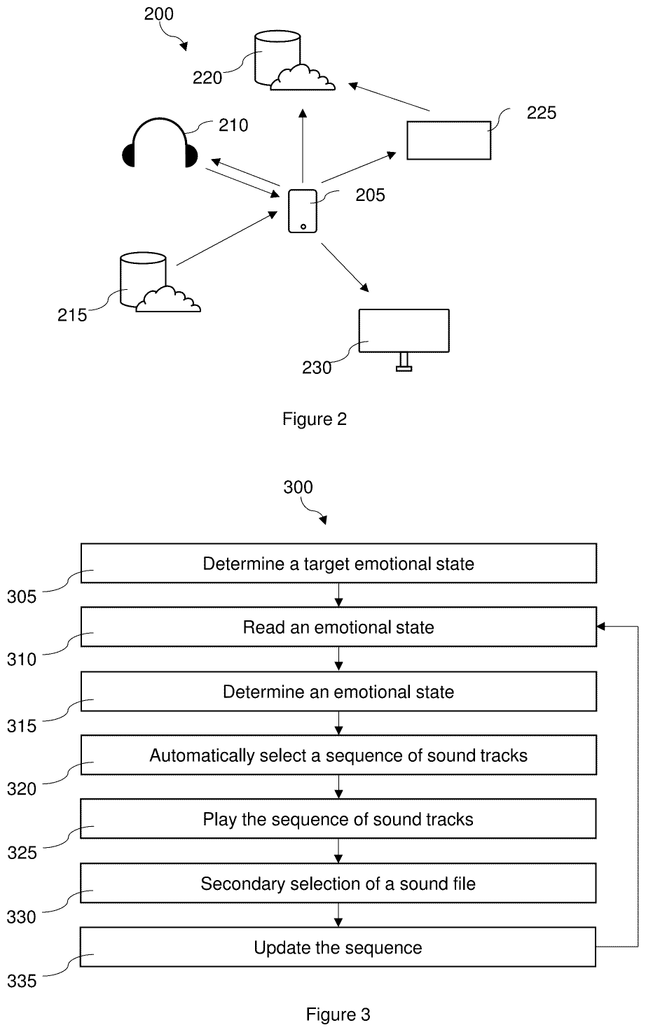

FIG. 2 shows, schematically, a particular embodiment of a technical ecosystem 200 enabling the implementation of the device 100 that is the subject of the present invention. This technical ecosystem 200 comprises:
a central terminal 205, an electronic calculation circuit configured to execute sequences of algorithmic instructions in the form of software, such as, for example, a computer, server or smartphone;
a database 215 of audio files accessible via the central terminal 205;
an EEG headset 210 equipped with earphones enabling the playback of audio files, connected to the central terminal 205 to:
receive an audio file to be played (or a location of a resource to be played on a data network); and
send to this terminal 205 an indicator representative of an EEG signal read, this terminal 205 determining locally an emotional state of the wearer of the headset 210, or transmitting this indicator to be determined remotely, by using a computer server for example;
a classification database 220 of emotional states associated to sound files, associated to a computer resource for calculation (unnumbered) configured to execute a classification algorithm, which database 220 can be fed as audio files are played by the central terminal 205 or directly connected to the database 215 of audio files;
an interface 225 for accessing the classification database 220, by the central terminal 205 (or another calculation resource), configured to produce, based on the target emotional state determined, a list of candidate sound files for inclusion in the sequence to be produced for modifying the emotional state of the user, or alternatively directly the sequence of sound files to be played; and
optionally, a monitoring and control interface 230 making it possible to display statistics, and an interface for the parameterisation of the device 100.

FIG. 3 shows, schematically, a particular embodiment of the method 300 that is the subject of the present invention. This method 300 for modifying an emotional state of a user comprises:
a step 305 of determining a target emotional state;
and then, iteratively, at least one portion of the following steps:
a step 310 of the real-time reading of electroencephalographic signals;
a step 315 of determining an emotional state based on an electroencephalographic signal read;
a step 320 of automatically selecting a sequence of at least one sound file, from a previously assembled list of sound files, based on the target emotional state determined, the electroencephalographic signal read, and at least one parameter associated to each said sound file; and
a step 325 of an electroacoustic transducer playing the selected sequence of sound files.

In some embodiments, such as that shown in FIG. 3, the method 300 also comprises:
a step 330 of a secondary selection of a sound file; and
a step 335 of updating the sequence based on the sound file selected manually by the secondary selector.

Examples of the implementation of steps of the method 300 are described with reference to the corresponding means, as described with reference to FIGS. 1 and 2.

Preferably, the means of the device 100 and the technical ecosystem 200 are configured to implement the steps of the method 300 and their embodiments as described above, and the method 300 and its different embodiments can be implemented by the means of the device 100 and/or the technical ecosystem 200.

1. A device for modifying an emotional state of a user, which comprises:
a real-time reader of electroencephalographic signals;
a module for determining an emotional state based on an electroencephalographic signal read;
a means for determining a target emotional state;
an automatic selector of a sequence of at least one sound file, from a previously assembled list of sound files, based on the target emotional state determined, the electroencephalographic signal read, and at least one parameter associated to each said sound file; and
an electroacoustic transducer configured to play the selected sequence of sound files;
characterised in that the device also comprises:
a secondary selector of a sound file; and
a means for updating the sequence based on the sound file selected manually by the secondary selector.

2. The device according to claim 1, which comprises:
a collector of sound file identifiers; and
a sound file classifier configured to associate, to at least one sound file identifier, a parameter representative of an emotional state fostered by that sound file.

3. The device according to claim 2, wherein the classifier is a trained machine learning system.

4. The device according to claim 3, wherein the trained machine learning system is a supervised neural network configured to receive, as an input layer, parameter values and, as an output layer, emotional state indicators corresponding to the input layer.

5. The device according to claim 3, wherein the classifier is configured to classify a sound file by assigning a value to at least one among three characteristics:
valence;
arousal; and
dominance;
at least one characteristic being utilized to determine an emotional state fostered by a sound track.

6. The device according to claim 3, wherein the machine learning system is also pre-trained by using a set of data not specific to the user.

7. The device according to claim 1, wherein at least one sound file is associated to an indicator of a behaviour of the user regarding each said sound file, the automatic selector being configured to select a sequence of at least one sound file based on a value of this indicator for at least one sound file.

8. The device according to claim 7, wherein the indicator of the user's behaviour is a parameter representative of a number of plays and/or of a number of playback interruptions in favour of another sound track.

9. The device according to claim 7, wherein the automatic selector comprises a sound file filter based on at least one indicator of a behaviour of the user regarding at least one sound file, the selector being configured to select a sound files sequence from a list of sound files filtered by the filter.

10. The device according to claim 1, wherein a parameter used by the automatic selector to select a sound files sequence is an indicator representative of an emotional state value associated to at least one sound file.

11. The device according to claim 10, wherein a parameter used by the automatic selector to select a sound files sequence is, in addition, a technical parameter chosen from the duration, mode, tonality, quantification of the beat and the tempo of the sound file.

12. The device according to claim 10, wherein the sound files sequence is configured to have a gradient of increasing emotional state value corresponding to the target emotional state determined.

13. The device according to claim 1, wherein the real-time reader of electroencephalographic signals is non-invasive.

14. The device according to claim 13, wherein the reader is an electroencephalogram type of headset.

15. A method for modifying an emotional state of a user, which comprises:
a step of determining a target emotional state;
and then, iteratively, at least one portion of the following steps:
a step of the real-time reading of electroencephalographic signals;
a step of determining an emotional state based on an electroencephalographic signal read;
a step of automatically selecting a sequence of at least one sound file, from a previously assembled list of sound files, based on the target emotional state determined, the electroencephalographic signal read, and at least one parameter associated to each said sound file;
a step of an electroacoustic transducer playing the selected sequence of sound files;
characterised in that the method also comprises:
a step of a secondary selection of a sound file; and
a step of updating the sequence based on the sound file selected manually by the secondary selector.
